(19) United States
(12) Patent Application Publication
(10) Pub. No.: US 2023/0414132 A1
(43) Pub. Date: Dec. 28, 2023

(54) SYSTEM AND METHOD FOR PROVIDING REHABILITATION IN A VIRTUAL ENVIRONMENT

(71) Applicant: Shanghai United Imaging Intelligence Co., LTD., Xuhui District (CN)

(72) Inventors: Abhishek Sharma, Cambridge, MA (US); Arun Innanje, Cambridge, MA (US); Benjamin Planche, Cambridge, MA (US); Meng Zheng, Cambridge, MA (US); Shanhui Sun, Cambridge, MA (US); Ziyan Wu, Cambridge, MA (US); Terrence Chen, Cambridge, MA (US)

(73) Assignee: Shanghai United Imaging Intelligence Co., LTD., Xuhui District (CN)

(21) Appl. No.: 17/849,109

(22) Filed: Jun. 24, 2022

(51) Int. Cl. A61B 5/11; A61B 5/00; G09B 19/00

(52) U.S. Cl. CPC A61B 5/1124; A61B 5/1113; A61B 5/749; A61B 5/7267; A61B 5/4848; G09B 19/003; A61B 2505/09; G06F 3/011

(57) ABSTRACT

A system for providing rehabilitation in a virtual environment includes an extended reality (XR) headset to present a first rehabilitation therapy to a patient in a virtual environment. A sensing device is configured to track physical movements of the patient and a processor is configured to receive the sensing data to determine pose information. The processor is configured to determine a performance metric associated with the physical movements and compare the performance metric with a reference metric to determine whether the patient has successfully performed the defined physical movements. The processor is configured to change the first rehabilitation therapy to a second rehabilitation therapy based on a difference between the performance metric and the reference metric upon determining that the patient has unsuccessfully performed the defined physical movements. The system aids the patient by changing the rehabilitation therapies according to the performance of the patient.

### TECHNICAL FIELD

The aspects of the disclosed embodiments relate generally to the field of virtual environment and more specifically, to a system and a method for providing rehabilitation in a virtual environment.

### BACKGROUND

With the rapid development in communication technology, a virtual environment, for example, a metaverse has become widespread. The virtual environment generally provides new ways to virtually connect as well as communicate with a plurality of users at a same time. The virtual environment is similar to a physical place, like a meeting room, a classroom, a museum, and the like.

One application of the virtual environment is for rehabilitation of a patient. The rehabilitation is care, which help patients to get back or improve abilities that are needed for daily life. The abilities include physical, mental, or cognitive, that are lost because of a disease, injury or due to side effect from a medical treatment.

A conventional rehabilitation virtual environment system is used present various rehabilitation therapies using smart mirrors or smart phones. However, the conventional rehabilitation virtual environment system only displays the rehabilitation therapies according to the sequence in which they are stored in a memory. If a patient is not able to perform the rehabilitation therapy, this poses a problem for the patient.

Therefore, in light of the foregoing discussion, there exists a need to overcome the aforementioned drawbacks associated with the conventional rehabilitation virtual environment system.

### SUMMARY

The aspects of the disclosed embodiments are directed to a system and a method for providing rehabilitation in a virtual environment. An aim of the disclosed embodiments is to provide an improved system and a method for dynamically changing the rehabilitation therapies in the virtual environment, according to the feedback of a patient.

One or more advantages of the disclosed embodiments are achieved by the solutions provided in the enclosed independent claims. Advantageous implementations of the present disclosure are further defined in the dependent claims.

The aspects of the disclosed embodiments provide a system for providing rehabilitation in a virtual environment. In one embodiment, the system comprises an extended reality (XR) headset configured to present a first rehabilitation therapy to a patient in the virtual environment. The system comprises a sensing device configured to track physical movements of the patient when the patient performs a first activity indicated by the first rehabilitation therapy. The system also comprises a hardware processor communicably coupled to the XR headset and the sensing device. The hardware processor is configured to receive sensing data from the sensing device, determine pose information associated with the first activity of the patient based on the received sensing data, and determine a performance metric associated with the physical movements of the patient in the first activity based on the determined pose information. Further, the hardware processor is configured to compare the performance metric with a reference metric to determine whether the patient has successfully performed one or more defined physical movements for a completion of the first activity indicated by the first rehabilitation therapy. Furthermore, the processor is configured to change the first rehabilitation therapy to a second rehabilitation therapy based on a difference between the performance metric and the reference metric upon determining that the patient has unsuccessfully performed the one or more defined physical movements for the first activity.

The disclosed system provides dynamically changing rehabilitation therapies according to the requirement of the patient. The system provides an improved way to treat the patient by evaluating the performance of the patient and changing the rehabilitation therapy accordingly. The dynamic change in the rehabilitation therapies disclosed in the system provides adequate rehabilitation therapy to the patient. Further, the system obtains a user input corresponding to a user-feedback associated with an effectiveness and an experience of the patient from the rehabilitation therapy and corresponding changes the rehabilitation therapy according to the capability of the patient. Alternatively stated, the system dynamically learns by continuously monitoring the patient and guide the patient through the journey of rehabilitation by constantly adapting and improving according to the requirements of the patient in order to achieve a final objective of attaining the required fitness.

It is to be appreciated that all the aforementioned implementation forms can be combined. It has to be noted that all devices, elements, circuitry, units, and means described in the present application could be implemented in the software or hardware elements or any kind of combination thereof. All steps which are performed by the various entities described in the present application, as well as the functionalities described to be performed by the various entities, are intended to mean that the respective entity is adapted to or configured to perform the respective steps and functionalities. Even if, in the following description of specific embodiments, a specific functionality or step to be performed by external entities is not reflected in the description of a specific detailed element of that entity that performs that specific step or functionality, it should be clear for a skilled person that these methods and functionalities can be implemented in respective software or hardware elements, or any kind of combination thereof. It will be appreciated that features of the present disclosure are susceptible to being combined in various combinations without departing from the scope of the present disclosure as defined by the appended claims.

Additional aspects, advantages, features, and objects of the present disclosure would be made apparent from the drawings and the detailed description of the illustrative implementations construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

The aspects of the disclosed embodiments will now be described, by way of example only, with reference to the following diagrams wherein:

FIG. 1A is a diagram illustrates an exemplary system for providing rehabilitation in a virtual environment, in accordance with the aspects of the disclosed embodiments;

FIG. 1B is a block diagram illustrating various exemplary components of a system for providing rehabilitation in a virtual environment, in accordance with the aspects of the disclosed embodiments;

FIG. 1C is a block diagram illustrating various exemplary components of an extended reality (XR) headset, in accordance with the aspects of the disclosed embodiments;

FIG. 2 is a diagram illustrating an implementation scenario of rehabilitation therapy in a virtual environment, in accordance with the aspects of the disclosed embodiments; and

FIGS. 3A to 3C collectively represent a flow chart of a method for providing rehabilitation in a virtual environment, in accordance with the aspects of the disclosed embodiments.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the aspects of the disclosed embodiments have been disclosed, those skilled in the art would recognize that other embodiments for carrying out or practicing the aspects of the disclosed embodiments are also possible.

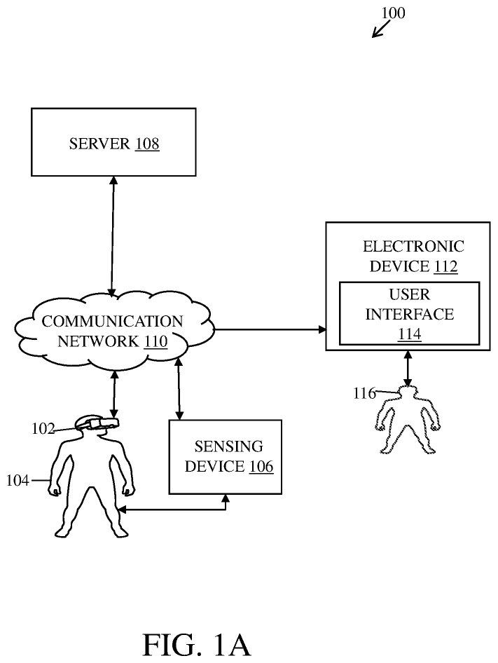

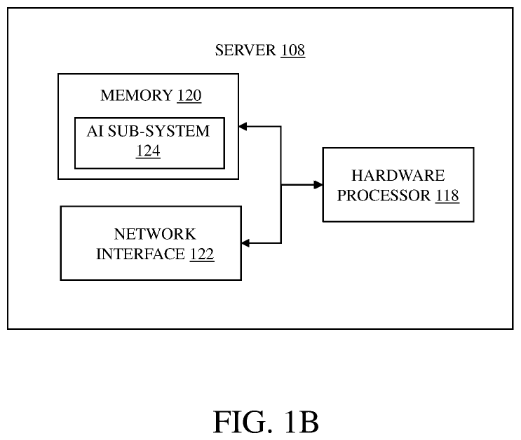

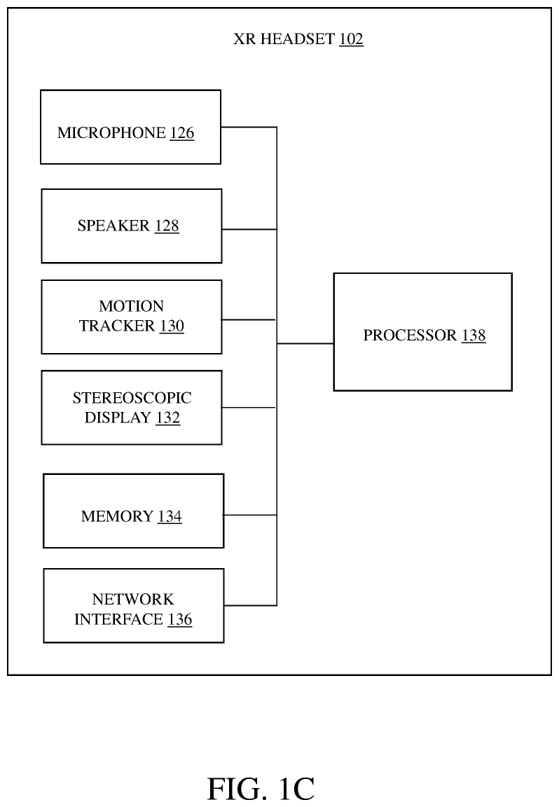

FIG. 1A is a diagram illustrating an exemplary system 100 for providing rehabilitation in a virtual environment, in accordance with the aspects of the disclosed embodiments. As is shown in FIG. 1A, the system 100 includes an extended reality (XR) headset 102 for accessing the virtual environment by a patient 104. There is further shown a sensing device 106 to sense one or more physical movements of the patient 104. Furthermore, there is shown a server 108 that is wirelessly connected to the XR headset 102 through a communication network 110.

In one embodiment, an electronic device 112 is connected to the communication network 110. The electronic device 112 is used to display the activities of the patient 104 to a care giver 116 through a user interface 114.

As illustrated in the example of FIGS. 1A and 1B, in one embodiment, the extended reality (XR) headset 102 is configured to present a first rehabilitation therapy to the patient in the virtual environment. The sensing device 106 is configured to track physical movements of the patient 104 when the patient 104 performs a first activity indicated by the first rehabilitation therapy.

In one embodiment, a hardware processor 118 of the server 108, shown in FIG. 1B, is communicably coupled to the XR headset 102 and the sensing device 106. The hardware processor 118 in this example is configured to receive sensing data from the sensing device 106 and determine pose information associated with the first activity of the patient 104 based on the received sensing data. In an implementation, the pose information is determined by an Artificial Intelligence (AI) sub-system provided in the server 108.

In one embodiment, the hardware processor 118 is further configured to determine a performance metric associated with the physical movements of the patient 104 during performance of the first activity based on the determined pose information. The hardware processor 118 can be further configured to compare the performance metric with a reference metric to determine whether the patient 104 has successfully performed one or more defined physical movements for a completion of the first activity indicated by the first rehabilitation therapy.

The hardware processor 118 can be configured to change the first rehabilitation therapy to a second rehabilitation therapy. In one embodiment, the change is based on determining that the patient has unsuccessfully performed the one or more defined physical movements for the first activity, and a difference between the performance metric and the reference metric.

The XR headset 102 may include suitable logic, circuitry, interfaces and/or code that is configured to enable the patient 104 to one or more view and interact with the virtual environment. The XR headset 102 provides a view of a combination of real and virtual environments that includes augmented reality (AR), virtual reality (VR), mixed reality (MR), and the areas interpolated among them.

The sensing device 106 may include suitable logic, circuitry, interfaces and/or code that is configured to one or more of detect and sense one or more physical movements of the patient 104. Further, the sensing device 106 may also be used to capture various images of the patient 104 from different orientations. Examples of the sensing device 106 may include, but are not limited to, a camera, an image sensor, a motion sensor, a pose sensor, and the like. In the system 100, one sensing device (i.e., the sensing device 106) is shown only for sake of simplicity. However, in another implementation, one or more sensing devices may be used.

The server 108 may include suitable logic, circuitry, interfaces, and/or code that is communicably coupled to the XR headset 102 through the communication network 110. Alternatively stated, the server 108 is configured to provide access of the virtual environment to the patient 104. The server 108 may be further configured to provide a live feed of the actions performed by the patient 104 in the virtual environment. Examples of implementation of the server 108 include, but are not limited to, a storage server, a cloud-based server, a web server, an application server, or a combination thereof. In an implementation, the server 108 may include the AI sub system.

The communication network 110 may include suitable logic, circuitry, and/or interfaces through which the XR headset 102, and the server 108 communicate with each other. Examples of the communication network 110 may include, but are not limited to, a cellular network (e.g., a 2G, a 3G, long-term evolution (LTE) 4G, a 5G, or 5G NR network, such as sub 6 GHz, cmWave, or mmWave communication network), a wireless sensor network (WSN), a cloud network, a Local Area Network (LAN), a vehicle-to-network (V2N) network, a Metropolitan Area Network (MAN), and/or the Internet.

The electronic device 112 may include suitable logic, circuitry, and/or interfaces that is used by the care giver 116 to monitor the performance of the patient 104. Examples of the electronic device 112 may include, but are not limited to, a computer, mobile phone, laptop, a display device, and the like.

The user interface 114 may include suitable logic, circuitry, and/or interfaces that is to represent a data related to the performance of the patient 104 on the electronic device 112. The user interface 114 is connected to the server 108 through the communication network 110 to receive the data related to the performance of the patient 104. Further, the user interface 114 is accessed by the care giver 116.

In operation, the patient 104 wears the XR headset 102 to access the virtual environment to perform a rehabilitation therapy, such as, jumping for ten minutes. The patient 104 performs the rehabilitation therapy by jumping in the real world, where the patient 104 is present. Further, the sensing device 106 senses the one or more physical movements of the patient 104 while performing the rehabilitation therapy and provides the sensed data to the server 108 through the communication network 110. Furthermore, the server 108 analyses the sensed data to check whether the patient 104 has performed the rehabilitation therapy successfully or not. Further, the server 108 also represents the analysed data about the performance of the patient 104 on the user interface 114 that is displayed on the electronic device 112, which helps the care giver 116 to monitor the performance of the patient 104. In an implementation, if the patient 104 jumped only for three minutes, then the server 108 analyses that the patient 104 has not performed the rehabilitation therapy successfully. Further the server 108 changes the rehabilitation therapy according to capabilities of the patient 104. Moreover, the care giver 116 also provide an input to the server 108 through the electronic device 112 to change the rehabilitation therapy according to the performance of the patient 104 in the first rehabilitation therapy. Thus, the system 100 dynamically learns by continuously monitoring the patient 104 and guiding the patient 104 through the journey of rehabilitation. The system 100 is configured to constantly adapt and improve according to the requirements of the patient 104 in order to achieve a final objective of attaining the required fitness or rehabilitation.

FIG. 1B is a block diagram that illustrates various exemplary components of a server used for providing rehabilitation in a virtual environment, in accordance with an embodiment of the present disclosure. FIG. 1B is described in conjunction with elements from FIG. 1A. With reference to FIG. 1B, there is shown a block diagram of the server 108 that includes a hardware processor 118, a memory 120, and a network interface 122. The server 108 may further include an Artificial Intelligence (AI) sub-system 124, for example, in the memory 120 of the server 108.

The hardware processor 118 may include suitable logic, circuitry, interfaces, or code that is configured to process an input (e.g., one or more physical movements of the patient 104) provided by the sensing device 106. Further, the hardware processor 118 is configured to analyse the performance of the patient 104 and provide suitable rehabilitation therapy from a plurality of rehabilitation therapies. In an implementation, the hardware processor 118 may lie in the server 108. In another implementation, the hardware processor 118 may be an independent unit and may lie outside the server 108. Examples of the hardware processor 118 may include, but are not limited to, a processor, a digital signal processor (DSP), a microprocessor, a microcontroller, a complex instruction set computing (CISC) processor, an application-specific integrated circuit (ASIC) processor, a reduced instruction set (RISC) processor, a very long instruction word (VLIW) processor, a state machine, a data processing unit, a graphics processing unit (GPU), and other processors or control circuitry. In an implementation, alternatively, the AI sub-system 124 may be implemented in the hardware processor 118.

The memory 120 may include suitable logic, circuitry, and/or interfaces that is configured to store the plurality of rehabilitation therapies required to be used by the hardware processor 118 and displayed to the patient 104 by the XR headset 102. In an implementation, the memory 120 may also be configured to store the data related to the performance of the patient 104 received from the sensing device 106. Examples of implementation of the memory 120 may include, but are not limited to, an Electrically Erasable Programmable Read-Only Memory (EEPROM), Dynamic Random-Access Memory (DRAM), Random Access Memory (RAM), Read-Only Memory (ROM), Hard Disk Drive (HDD), Flash memory, a Secure Digital (SD) card, Solid-State Drive (SSD), and/or CPU cache memory.

The network interface 122 may include suitable logic, circuitry, and/or interfaces that is configured to communicate with the XR headset 102 and the electronic device 112. Examples of the network interface 122 include, but are not limited to, a data terminal, a transceiver, a facsimile machine, and the like.

The AI sub-system 124 may include suitable logic, circuitry, and/or interfaces that may be referred to as one or more smart modules capable to perform tasks automatically that typically require human intelligence. Further, the AI sub-system 124 learns from the performance of the patient 104 and assist the hardware processor 118 to adapt according to the capabilities of the patient 104 and choose more suitable rehabilitation therapy for the patient 104.

The server 108 is connected to the XR headset 102 through the communication network 110 in order to provide access of the virtual environment to the patient 104. Further, there are plurality of rehabilitation therapies that are stored in the memory 120. Furthermore, a rehabilitation therapy from the plurality of rehabilitation therapies is fetched from the memory 120 by the hardware processor 118 and sent to the XR headset 102. The XR headset 102 is connected to the hardware processor 118 through the network interface 122. Further, the XR headset 102 enables the patient 104 to view the rehabilitation therapy received by the hardware processor 118. Further, the hardware processor 118 analyses the data received from the sensing device 106 through the network interface 122 about the movement of the patient 104 while performing one of the rehabilitation therapies. Moreover, the AI sub-system 124 learns about the capabilities of the patient 104 by the data received from the sensing device 106. Furthermore, the AI sub-system 124 assist the hardware processor 118 in changing the rehabilitation therapy according to the performance of the patient 104.

FIG. 1C is a block diagram that illustrates various exemplary components of an extended reality (XR) headset 102, in accordance with an embodiment of the present disclosure. FIG. 1C is described in conjunction with elements of FIGS. 1A and 1B. With reference to FIG. 1C, there is shown a block diagram of the extended reality (XR) headset 102 that includes a microphone 126, a speaker 128, a motion tracker 130, a stereoscopic display 132, a memory 134 a network interface 136, and a processor 138.

The microphone 126 may include suitable logic, circuitry, and/or interfaces that may be referred to as an audio capture component that is used in the XR headset 102. The audio capture component is used to capture the feedback of the patient 104 by receiving an audio input. Generally, the microphone 126 converts the audio input (i.e., the feedback of the patient 104 in the audio form) to an electrical signal that is sent to the processor 138 for further processing. Moreover, the speaker 128 may include suitable logic, circuitry, and/or interfaces that may be referred to as an audio output component that is used in the XR headset 102 to provide an audio output related to the rehabilitation therapy to the patient 104. Generally, the speaker 128 is configured to convert electrical signals to the audio output.

The motion tracker 130 may include suitable logic, circuitry, and/or interfaces that is configured to is used in the XR headset 102 to track the one or more physical movements (e.g., physical movements of legs and hands) of the patient 104. After tracking the movement, the motion tracker 130 generates electrical signals that are sent to the processor 138 for further processing. In addition, the stereoscopic display 132 may include suitable logic, circuitry, and/or interfaces that is configured to provide a three-dimensional (3D) view of the virtual environment to the patient 104. The stereoscopic display 132 is configured to convey depth perception to the patient 104 by means of stereopsis for binocular vision.

The memory 134 may include suitable logic, circuitry, and/or interfaces that is configured to store rehabilitation therapies used by the processor 138 and displayed by the XR headset 102 to the patient 104. The memory 134 may also be configured to store the data received from the sensing device 106. Examples of implementation of the memory 134 corresponds to examples of the memory 120 (of FIG. 1B). The memory 134 may store an operating system or other program products (including one or more operation algorithms) that is operated by the processor 138.

The network interface 136 may include suitable logic, circuitry, and/or interfaces that is configured to enable the XR headset 102 to communicate with the server 108. Examples of the network interface 136 corresponds to the examples of the network interface 122 (of FIG. 1B).

The processor 138 may include suitable logic, circuitry, interfaces, or code that is configured to process an input provided by the sensing device 106. Further, the processor 138 may also be configured to analyse the performance of the patient 104 and provide suitable rehabilitation therapy from a plurality of rehabilitation therapies. In an implementation, the processor 138 may correspond to the hardware processor 118 of the FIG. 1B. Examples of the processor 138 may include, but are not limited to, a processor, a digital signal processor (DSP), a microprocessor, a microcontroller, a complex instruction set computing (CISC) processor, an application-specific integrated circuit (ASIC) processor, a reduced instruction set (RISC) processor, a very long instruction word (VLIW) processor, a state machine, a data processing unit, a graphics processing unit (GPU), and other processors or control circuitry.

There is provided the system 100 of FIG. 1A for rehabilitation in the virtual environment. The rehabilitation is a care provided in the virtual environment by means of a plurality of rehabilitation therapies which are displayed to the patient 104 for treatment of a medical disease, or a trauma and the like. Further, the patient 104 is required to perform one or more physical movements in a real world according to the instructions provided by the rehabilitation therapy in the virtual environment.

In accordance with an embodiment, the system 100 includes the virtual environment that is a metaverse. The metaverse (e.g., a virtual model) may be designed through any suitable 3D modelling technique and computer assisted drawings (CAD) methods that enables exploration thereof and communications between users in the metaverse. Thus, the metaverse may be a virtual place or collaboration of multiple virtual platforms including a virtual setting, where one or more users may walk around, perform various activities, and give feedback to each other virtually.

The system 100 includes the extended reality (XR) headset 102 that is configured to present a first rehabilitation therapy to a patient 104 in the virtual environment. In an implementation, the XR headset 102 is used to provide a visual representation of a plurality of rehabilitation therapies to the patient 104. Further, the XR headset 102 initially present the first rehabilitation therapy (e.g., jumping) from amongst the plurality of rehabilitation therapies to the patient 104. The rehabilitation therapies presented by the XR headset 102 may include, but are not limited to, walking, jumping, running, motivational speech, instructions to perform an activity, and the like.

The system 100 further includes the sensing device 106 that is configured to track physical movements of the patient 104 when the patient 104 performs a first activity indicated by the first rehabilitation therapy. The sensing device 106 may include a plurality of sensors, such as a plurality of cameras, a plurality of motion sensors, a plurality of pose sensors, and the like. In an implementation, the first rehabilitation therapy may correspond to running. In such implementation scenario, the patient 104 performs running in the real world, then one motion sensor from the plurality of motion sensors tracks the movements of legs of the patient 104 and another motion sensor from the plurality of motion sensors tracks the movements of an upper portion of the body of the patient 104. Alternatively stated, the sensing device 106 is used to track the one or more physical movements of the patient 104. In another implementation, the first rehabilitation therapy may correspond to push-ups. In such implementation scenario, the patient 104 performs push-ups in the real world, then the sensing device 106 collectively track one or more physical movements of the whole body of the patient 104. In an example, one image sensor the plurality of image sensors captures real-time images that includes red-green-blue (RGB), depth, thermal, infra-red image of the whole body of the patient 104 and one motion sensor of the plurality of motion sensors senses motion of the whole body of the patient 104. As a result, the sensing device 106 collectively tracks the physical movements of the patient 104 that performs the first activity in the real world by viewing and listing to instructions provided in the first rehabilitation therapy in the virtual environment.

The system 100 further includes the hardware processor 118 communicably coupled to the XR headset 102 and the sensing device 106. Moreover, the hardware processor 118 is configured to receive sensing data from the sensing device 106. In an implementation, the hardware processor 118 is configured to receive the sensing data from the sensing device 106. The sensing data may include the position of the patient 104, movement of various body parts (e.g., legs, hands, etc.) of the patient 104, body temperature of the patient 104, orientation of the patient 104, and the like. In an example, the first rehabilitation therapy corresponds to running, in that case, the hardware processor 118 receives sensing data that includes movement of the legs the patient 104, movement of the upper portion of the body of the patient 104 and real-time images of the patient 104 while performing the first activity. In another example, the first rehabilitation therapy may correspond to push-ups, in that case, the hardware processor 118 receives the sensing data that includes movement of the torso of the patient 104, movement of the elbows of the patient 104 and real-time images of the patient 104 while performing the first activity.

The hardware processor 118 is further configured to determine pose information associated with the first activity of the patient 104 based on the received sensing data. Further the hardware processor 118 uses the AI sub-system 124 to determine the pose information required to evaluate the performance of the patient 104 while performing the first activity. The AI sub-system 124 is configured to evaluate the sensing data in order to determine the pose information.

In accordance with an embodiment, the pose information comprises one or more of a position of the patient 104, an orientation of the patient 104 in a three-dimensional (3D) space, a joint position of the patient 104 in the 3D space, or other pose information. The pose information includes position of the patient 104, movement of joints of the body of the patient 104, rotating angle of joints of the body of the patient 104 and the like. For example, if the first rehabilitation therapy indicates the first activity, such as running and the patient 104 performs the physical movement in the real world, then the hardware processor 118 receives the sensing data for pose determination. The pose determination includes an angular movement of the knee joint of the patient 104, the angular movement of the ankle joint of the patient 104, an orientation of a neck of the patient 104 and the like. In another example, if the first rehabilitation therapy indicates the first activity, such as push-ups and the patient 104 performs the physical movement in the real world, then the hardware processor 118 uses the AI sub-system 124 to determine pose information that includes an angular movement of shoulders of the patient 104, the angular movement of elbow joints of the patient 104, the angular movement of a wrist of the patient 104, orientation of a neck of the patient 104, and the like.

The hardware processor 118 is further configured to determine a performance metric associated with the physical movements of the patient 104 during performance of the first activity based on the determined pose information. The performance metric is the evaluation of the physical movements performed by the patient 104 with respect to the movements required to complete the first activity indicated by the first rehabilitation therapy. The performance metric is determined by the hardware processor 118 based on the pose information determined by the AI sub-system 124.

The hardware processor 118 is further configured to compare the performance metric with a reference metric to determine whether the patient 104 has successfully performed one or more defined physical movements for a completion of the first activity indicated by the first rehabilitation therapy. The reference metric includes the predefined values of performance parameters related to one or more rehabilitation therapies that indicates successful implementation of the one or more rehabilitation therapies stored in the memory 120 of the server 108 of FIGS. 1A and 1B. The performance metric is compared with the reference metric to evaluate the performance of the patient 104 and determine whether the patient 104 has successfully performed the first activity or not.

For example, if the first rehabilitation therapy indicates the first activity is hands-up, then the performance metric may define the angles of rotation of hands of the patient 104 while the first activity is performed. The reference metric defines the required angle of rotation of hands to complete the first activity. Thereafter, the hardware processor 118 compares the angle of rotation of hands of the patient 104 with the required angle of rotation of hands to determine whether the patient 104 has successfully performed one or more defined physical movements for the completion of the first activity or not.

In an implementation, the comparison of the performance metric with the reference metric may be used to determine a score for the performance of the patient 104. In an example, if the first rehabilitation therapy indicates the first activity, such as running, and the patient 104 starts walking in the real world, then in such case, the performance metric shows a low score. In another example, if the first rehabilitation therapy indicates the first activity, such as running, and the patient 104 starts running in the real world, then the performance metric shows a high score. As a result, the performance metric is beneficial to monitor an improvement in behaviour of the patient 104.

The hardware processor 118 is further configured to change the first rehabilitation therapy to a second rehabilitation therapy based on a difference between the performance metric and the reference metric upon determining that the patient 104 has unsuccessfully performed the one or more defined physical movements for the first activity. For example, the first rehabilitation therapy indicates the first activity, such as to move five steps forward, and the patient 104 moves only one step forward in the real world. In this scenario, the difference between the performance metric and the reference metric is large. The hardware processor 118 is configured to change the first rehabilitation therapy to the second rehabilitation therapy that is comfortable for the patient 104.

In accordance with an embodiment, the hardware processor 118 is further configured to quantify a progress of the patient 104 in one or more of the first rehabilitation therapy, the second rehabilitation therapy, or from the first rehabilitation therapy to the second rehabilitation therapy. The progress of the patient 104 is quantified by the hardware processor 118 by analysing the performance of the patient 104.

In an implementation, the progress is quantified by analysing the difference between the performance metric and reference metric after completion of the first rehabilitation therapy by the patient 104. In another implementation, the progress is quantified by analysing the difference between the performance metric and reference metric after completion of the second rehabilitation therapy by the patient 104. In a yet another implementation, the progress is quantified by analysing the difference between the performance metric of the first rehabilitation therapy and the performance metric of the second rehabilitation therapy.

In accordance with an embodiment, the hardware processor 118 is further configured to update a current rehabilitation therapy to a new rehabilitation therapy, based on the quantified progress of the patient 104 in the rehabilitation. Moreover, the current rehabilitation therapy is one of the first rehabilitation therapy or the second rehabilitation therapy. For example, if the patient 104 completes the first rehabilitation therapy, then the hardware processor 118 is configured to compare the performance metric and the reference metric associated with the first rehabilitation therapy. The hardware processor 118 is configured to quantify the progress of the patient 104, which in this case shows unsuccessful completion of the first rehabilitation therapy.

Further, the hardware processor 118 is configured to change the first rehabilitation therapy to the second rehabilitation therapy. However, if the patient 104 unsuccessfully performs the second rehabilitation therapy, then the hardware processor 118 is further configured to change the second rehabilitation therapy to the new rehabilitation therapy.

In accordance with an embodiment, the hardware processor 118 is further configured to obtain a user input corresponding to a user-feedback associated with an effectiveness and an experience of the patient 104 from the first rehabilitation therapy or the second rehabilitation therapy. The user-feedback is about the experience of the patient 104 after completing one of the rehabilitation therapies, such as the first rehabilitation therapy or the second rehabilitation therapy. In an implementation, the patient 104 completes the first rehabilitation therapy and provides the user-feedback (e.g., an audio feedback) to the hardware processor 118. In another implementation, the patient 104 completes the second rehabilitation therapy and provides the user-feedback to the hardware processor 118. The user-feedback provided by the patient 104 may be in form of an audio, a video, or a physical movement, and the like.

In accordance with an embodiment, the hardware processor 118 is further configured to update the second rehabilitation therapy to a third rehabilitation therapy based on the obtained input. Further, the input is one or more of a gesture-based input, a voice-based input, or an input received via an input device that is communicatively coupled to the XR headset 102. After completion of one of the rehabilitation therapies, such as the first rehabilitation therapy or the second rehabilitation therapy, the patient 104 provides the user-feedback to the hardware processor 118 by use of the input device. Examples of the input device may include, but are not limited to a microphone (e.g., the microphone 126), a laptop, a mobile phone, a computer, a joystick and the like.

Based on the user-feedback, the hardware processor 118 is configured to further update the rehabilitation therapy according to the requirements of the patient 104. For example, if the patient 104 performs the first rehabilitation therapy that corresponds to walking for ten minutes and after completing the first rehabilitation therapy, the patient 104 provides the user-feedback mentioning high difficulty. This can mean that the patient 104 feels difficulty in performing the first activity indicated by the first rehabilitation therapy. Thereafter, the hardware processor 118 receives the user-feedback and updates the first rehabilitation therapy to the second rehabilitation therapy that corresponds to walking for five minutes from ten minutes as indicated in the first rehabilitation therapy.

After completing the second activity related to the second rehabilitation therapy, the patient 104 provides the user-feedback mentioning low difficulty that means the second rehabilitation therapy is below the capability of the patient 104. Thereafter, the hardware processor 118 is configured to update the second rehabilitation therapy to the third rehabilitation therapy that corresponds to walking for seven minutes from five minutes as indicated in the second rehabilitation therapy. Therefore, the hardware processor 118 is configured to change the rehabilitation therapies until they are not according to requirements of the patient 104.

In accordance with an embodiment, the system 100 further comprises the artificial intelligence (AI) sub-system 124. In an implementation, the AI sub-system 124 is provided in the server 108. Moreover, the hardware processor 118 is further configured to learn, by use of the AI sub-system 124, a performance gap specific to the patient 104. The performance gap can be learned from movement information corresponding to the tracked physical movements of the patient 104. This can include when the patient 104 performs the first activity and the second activity indicated by the first rehabilitation therapy and the second rehabilitation therapy, respectively.

For example, after completion of the first activity as indicated by the first rehabilitation therapy, the AI sub-system 124 analyses the performance of the patient 104. Further, the patient 104 performs the second activity as indicated by the second rehabilitation therapy and after completion of the second activity, the AI sub-system 124 again analyses the performance of the patient 104 in the second activity. By virtue of the analysis performed by the AI sub-system 124, the hardware processor 118 is configured to compare the performance of the patient 104 in the first activity with the second activity to obtain the performance gap. The use of AI sub-system 124 leads to a more accurate estimation of the performance gap by the hardware processor 118 and makes the system 100 more adaptive in nature according to requirements of the patient 104.

In accordance with an embodiment, the hardware processor 118 is further configured to dynamically update a current rehabilitation therapy to a new rehabilitation therapy, based on the performance gap learnt by the AI sub-system 124. The current rehabilitation therapy is one of the first rehabilitation therapy or the second rehabilitation therapy.

For example, the performance gap of the patient 104 shows that the patient 104 has performed the first rehabilitation therapy unsuccessfully. In such scenario, the AI sub-system 124 learns from the performance gap and the hardware processor 118 is configured to dynamically change the first rehabilitation therapy to the second rehabilitation therapy. The second rehabilitation therapy can be somewhat easier to perform with respect to the first rehabilitation therapy.

In accordance with an embodiment, the hardware processor 118 is further configured to present the user interface 114 to the electronic device 112 associated with the care giver 116 of the patient 104. Examples of the electronic device 112 may include, a mobile phone, a monitor, a laptop, and other similar devices. Further, the user interface 114 is presented on the electronic device 112, which enables the care giver 116 of the patient 104 to monitor the performance of the patient 104. Moreover, the user interface 114 provides the facility to the care giver 116 to change the rehabilitation therapy according to the capability of the patient 104, such as by providing inputs to the hardware processor 118 through the user interface 114.

In accordance with an embodiment, the hardware processor 118 is further configured to obtain one or more new rehabilitation therapies based on one or more input received via the user interface 114. In one embodiment, the one or more inputs comprises a plurality of defined parameters to configure the AI sub-system 124 to define the one or more new rehabilitation therapies.

For example, if the patient 104 performs the first rehabilitation therapy that includes running for twenty minutes and the patient 104 is unsuccessful, then the hardware processor 118 changes the first rehabilitation therapy to the second rehabilitation therapy. In this example, the second rehabilitation therapy includes running for five minutes. However, if the care giver 116 provides the one or more inputs to the hardware processor 118 through the user interface 114 to define the parameter of running as ten minutes, then the hardware processor 118 changes the first rehabilitation therapy to the one or more new rehabilitation therapies that corresponds to running for ten minutes.

The system 100 provides dynamically changing rehabilitation therapies according to the requirements of the patient 104. The system 100 provides an improved way to treat the patient 104 by evaluating the performance of the patient 104 and changing the rehabilitation therapy accordingly. The dynamic change in the rehabilitation therapies manifested in the system 100 provides an adequate rehabilitation therapy to the patient 104.

Further, the system 100 obtains the user input corresponding to the user-feedback associated with an effectiveness and an experience of the patient 104 from the rehabilitation therapy and changes the rehabilitation therapy according to the capability of the patient 104. Alternatively stated, the system 100 dynamically learns by continuously monitoring the patient 104 and guiding the patient 104 through the journey of rehabilitation by constantly adapting and improving according to the requirements of the patient 104 in order to achieve a final objective of attaining the required fitness.

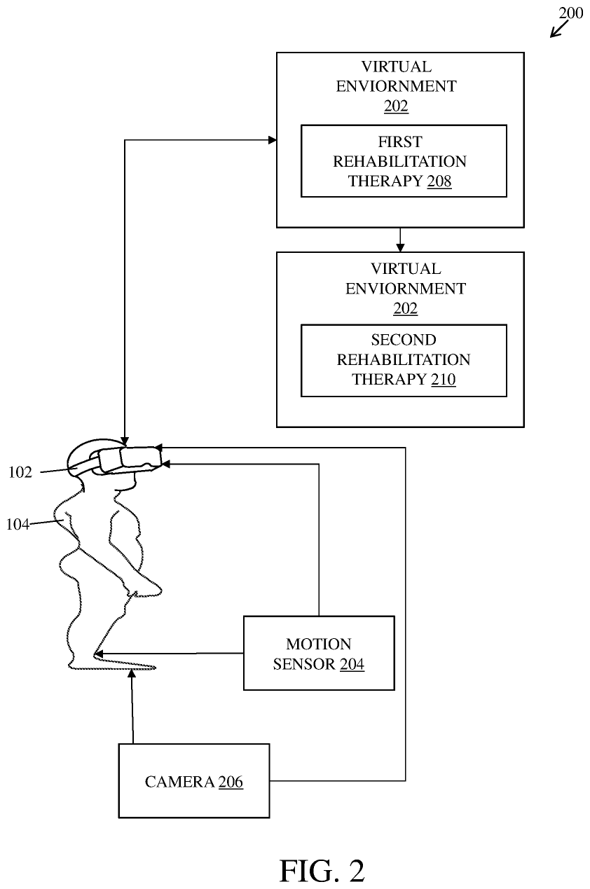

FIG. 2 is a diagram that illustrates an implementation scenario of one or more rehabilitation therapies in a virtual environment, in accordance with an embodiment of the present disclosure. FIG. 2 is described in conjunction with elements from FIG. 1A, FIG. 1B and FIG. 1C.

With reference to FIG. 2, there is shown an implementation scenario 200 of one or more rehabilitation therapies in a virtual environment 202. There is further shown the extended reality (XR) headset 102 that is configured to provide access of the virtual environment 202 to the patient 104. Furthermore, there is shown a motion sensor 204 and a camera 206 to track physical movements of the patient 104. Moreover, the virtual environment 202 includes a first rehabilitation therapy 208 and a second rehabilitation therapy 210.

In the implementation scenario 200, the XR headset 102 is configured to present the first rehabilitation therapy 208 to the patient 104 in the virtual environment 202. Thereafter, the patient 104 is required to perform the first activity indicated by the first rehabilitation therapy 208.

For example, the first rehabilitation therapy 208 indicates the first activity, such as to jump four feet. The patient 104 is then required to jump in the real world in order to complete the first activity indicated by the first rehabilitation therapy 208. When the patient 104 is performing the first activity, the motion sensor 204 captures the movement of the patient 104 and the camera 206 captures one or more images of the patient 104. Each of the camera 206 and the motion sensor 204 acts as the sensing device 106 (of FIG. 1A). Alternatively stated, each of the camera 206 and the motion sensor 204 is used to sense data (e.g., one or more physical movements) of the patient 104.

In one embodiment, the sensed data collected by each of the motion sensor 204 and the camera 206 is sent to the hardware processor 118 of the server 108. Furthermore, the hardware processor 118 uses the AI sub-system 124 to analyse the received data to determine a pose information. The pose information can include movement of knees of the patient 104, movement of legs of the patient 104, body posture of the patient 104, and the like.

In one embodiment, the hardware processor 118 is configured to determine a performance metric by evaluating the pose information of the patient 104 through the AI sub-system 124. The performance metric is further compared with a reference metric stored in the memory 120 of the server 108.

For, example, if the patient 104 successfully performs the first activity indicated by the first rehabilitation therapy 208, by jumping four feet in the real world, then the result after comparison shows a high score in the performance metric. If the patient 104 unsuccessfully performs the first activity indicated by the first rehabilitation therapy 208, by for example jumping only one foot in the real world, then a result after comparison shows a low score in the performance metric. As a result, the AI sub-system 124 of the server 108 learns capabilities of the patient 104.

The AI sub-system can simultaneously communicate to the hardware processor 118 to change the first rehabilitation therapy 208 to the second rehabilitation therapy 210, which may correspond more closely to the capabilities of the patient 104. In this example, the patient 104 may successfully perform the second activity indicated by the second rehabilitation therapy 210. Thus, the implementation scenario 200 indicates that the rehabilitation therapies can be changed depending on abilities of the patient 104 which further enables the patient 104 to achieve the fitness more efficiently.

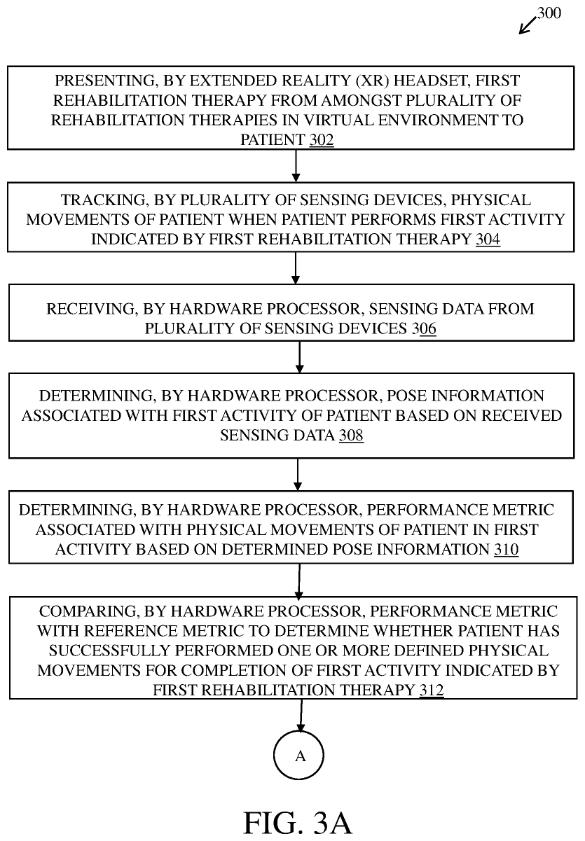

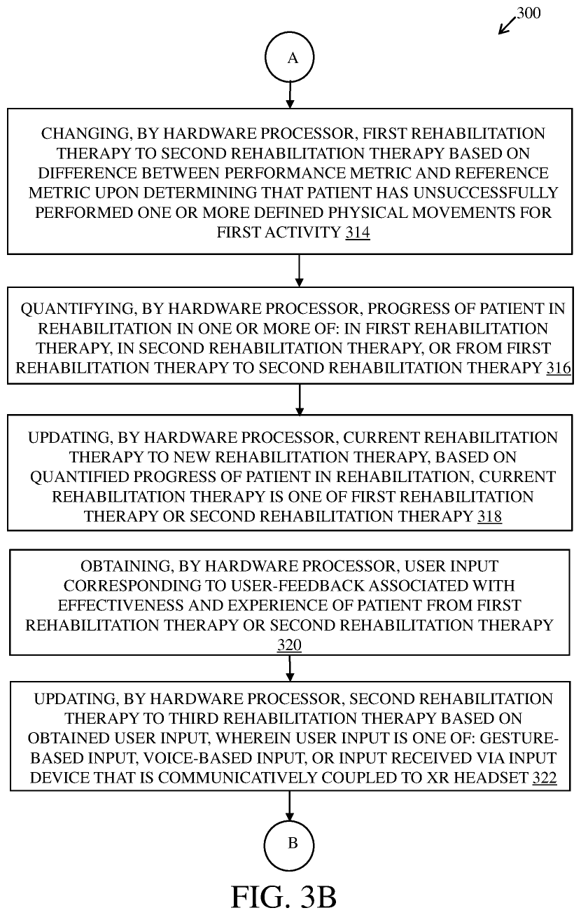

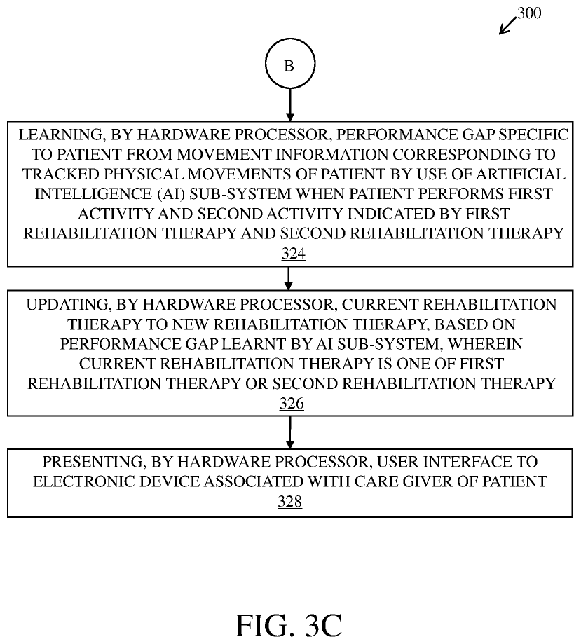

FIGS. 3A to 3C collectively represent a flow chart of a computer implemented method for providing rehabilitation in a virtual environment, in accordance with an embodiment of the present disclosure. FIGS. 3A to 3C are described in conjunction with elements of FIG. 1A, FIG. 1B, and FIG. 1C.

With reference to FIGS. 3A-3C, the flowchart of the computer implemented method 300 includes steps 302, 304, 306, 308, 310, 312, 314, 316, 318, 320, 322, 324, 326, and 328. The steps 302 to 312 are shown in FIG. 3A, the steps 314 to 322 are shown in FIG. 3B, and the steps 324 to 328 are shown in FIG. 3C.

The method 300 provides multiple rehabilitation therapies in the virtual environment that are performed by a patient 104 in a real world according to the instructions provided in the rehabilitation therapy in the virtual environment. The performance of the patient 104 is analysed and the rehabilitation therapy is changed accordingly.

Referring to FIG. 3A, step 302 includes presenting, by the extended reality (XR) headset 102, a first rehabilitation therapy to the patient 104 in the virtual environment. In one embodiment, the XR headset 102 is configured to initially present the first rehabilitation therapy from among a plurality of rehabilitation therapies to the patient 104.

At 304, the method 300 includes tracking, by the sensing device 106, physical movements of the patient 104 when the patient 104 performs a first activity indicated by the first rehabilitation therapy. In an implementation, the sensing device 106 includes image sensors, motion sensors and the like. Further, the image sensor may capture images that includes red-green-blue (RGB), depth, thermal, infra-red and the like.

At 306, the method 300 includes receiving, by a hardware processor 118, sensing data from the sensing device 106. The sensing data can include a position of the patient 104, movement of the body part of the patient 104, body temperature of the patient 104, orientation of the patient 104, and the like. In an implementation, the hardware processor 118 receives the sensing data from multiple sensing devices.

At 308, the method 300 includes determining, by the hardware processor 118, pose information associated with the first activity of the patient 104 based on the received sensing data. The hardware processor 118 uses the AI sub-system 124 to determine the pose information by evaluating the sensing data. The pose information includes the movement of the body structure of the patient 104. The pose information does not include movement of any object near the patient 104 and only provides information about a skeletal movement of the patient 104.

At 310, the method 300 includes determining, by the hardware processor 118, a performance metric associated with the physical movements of the patient 104 in the first activity based on the determined pose information. The performance metric determined by the hardware processor 118 by the help of pose information. The performance metric is the evaluation of the physical movements performed by the patient 104 with respect to the movements required to complete the first activity indicated by the rehabilitation therapy.

At 312, the method 300 includes comparing, by the hardware processor 118, the performance metric with a reference metric to determine whether the patient 104 has successfully performed one or more defined physical movements for a completion of the first activity indicated by the first rehabilitation therapy. The performance metric that is stored in the memory 120 is compared with the reference metric to check the performance of the patient 104. The performance metric also helps to monitor the health of the patient 104.

Referring to FIG. 3B, at 314, the method 300 includes changing, by the hardware processor 118, the first rehabilitation therapy to a second rehabilitation therapy. The change can be based on a determined difference between the performance metric and the reference metric upon determining that the patient 104 has unsuccessfully performed the one or more defined physical movements for the first activity. A change in the level of difficulty of the first rehabilitation therapy compared to the second rehabilitation therapy is based on the difference in the performance metric and the reference metric.

At 316, the method 300 includes quantifying, by the hardware processor 118, a progress of the patient 104 in the rehabilitation in one or more of: the first rehabilitation therapy, the second rehabilitation therapy, or from the first rehabilitation therapy to the second rehabilitation therapy. The hardware processor 118 is configured to analyse the performance of the patient 104 to quantify the progress of the patient 104. The progress is quantified by analysing performance of the patient 104 after the rehabilitation therapy or by comparing performance in one rehabilitation therapy to the performance in another rehabilitation therapy.

At 318, the method 300 includes updating, by the hardware processor 118, a current rehabilitation therapy to a new rehabilitation therapy, based on the quantified progress of the patient 104 in rehabilitation. The current rehabilitation therapy can be one of the first rehabilitation therapy or the second rehabilitation therapy. The rehabilitation therapy is updated according to the performance of the patient 104. In an implementation, the level, challenge or difficult of the new activity decreases as the performance of the patient 104 decreases. In an implementation, the level, challenge or difficulty of the new activity increases as the performance of the patient 104 increases.

At 320, the method 300 includes obtaining, by the hardware processor 118, an input corresponding to a user-feedback associated with an effectiveness and an experience of the patient 104 from the first rehabilitation therapy or the second rehabilitation therapy. The user-feedback is about the experience of the patient 104 after completing the rehabilitation therapy.

At 322, the method 300 includes updating, by the hardware processor 118, the second rehabilitation therapy to a third rehabilitation therapy based on the obtained input. In this example, the user input can be one or more of a gesture-based input, a voice-based input, or an input received via an input device that is communicatively coupled to the XR headset. The patient 104 provides the user-feedback after completing the rehabilitation therapy and the hardware processor 118 updates the rehabilitation therapy accordingly.

Referring to FIG. 3C, at 324, the method 300 includes learning, by the hardware processor 118, a performance gap specific to the patient 104. The performance gap is learned from movement information corresponding to the tracked physical movements of the patient 104 by use of the artificial intelligence (AI) sub-system 124 when the patient 104 performs the first activity and the second activity indicated by the first rehabilitation therapy and the second rehabilitation therapy. To obtain the performance gap, the AI sub-system 124 of the server 108 compares the performance of the patient 104 in the first rehabilitation activity with the performance in the second rehabilitation therapy.

At 326, the method 300 includes updating, by the hardware processor 118, a current rehabilitation therapy to a new rehabilitation therapy, based on the performance gap learnt by the AI sub-system 124. In this example, the current rehabilitation therapy is one of the first rehabilitation therapy or the second rehabilitation therapy.

In an implementation, the level, challenge or difficulty of the new activity decreases as the performance gap of the patient 104 increases. In an implementation, the level, challenge or difficulty of the new activity increases as the performance gap of the patient 104 decreases.

At 328, the method 300 includes presenting, by the hardware processor 118, a user interface 114 on an electronic device associated with a care giver 116 of the patient 104. The user interface 114 is presented on the electronic device to help the care giver 116 of the patient 104 monitor the performance of the patient 104. The user interface 114 enables the care giver 116 to change the plurality of rehabilitation therapies according to the capability of the patient 104 by providing inputs to the hardware processor 118.

The method 300 provides the facility to change rehabilitation therapies according to the capability of the patient 104. Further, the method 300 provides an improved way to treat the patient 104 by evaluating the performance of the patient 104 and changing the rehabilitation therapy accordingly. Furthermore, the method 300 obtains the user input corresponding to the user-feedback associated with an effectiveness and an experience of the patient 104 after performing the one of the plurality of rehabilitation therapies.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable combination or as suitable in any other described embodiment of the disclosure.

1. A system for providing rehabilitation in a virtual environment, the system comprising:
an extended reality (XR) headset configured to present a first rehabilitation therapy to a patient in the virtual environment;
a sensing device configured to track physical movements of the patient when the patient performs a first activity indicated by the first rehabilitation therapy; and
a hardware processor communicably coupled to the XR headset and the sensing device, wherein the hardware processor is configured to:
receive sensing data from the sensing device;
determine pose information associated with the first activity of the patient based on the received sensing data;
determine a performance metric associated with the physical movements of the patient during performance of the first activity based on the determined pose information;
compare the performance metric with a reference metric to determine whether the patient has successfully performed one or more defined physical movements for a completion of the first activity indicated by the first rehabilitation therapy; and
change the first rehabilitation therapy to a second rehabilitation therapy based on a difference between the performance metric and the reference metric upon determining that the patient has unsuccessfully performed the one or more defined physical movements for the first activity.

2. The system according to claim 1, wherein the hardware processor is further configured to quantify a progress of the patient in the rehabilitation in one or more of: in the first rehabilitation therapy, in the second rehabilitation therapy, or from the first rehabilitation therapy to the second rehabilitation therapy.

3. The system according to claim 2, wherein the hardware processor is further configured to update a current rehabilitation therapy to a new rehabilitation therapy, based on the quantified progress of the patient in the rehabilitation, wherein the current rehabilitation therapy is one of the first rehabilitation therapy or the second rehabilitation therapy.

4. The system according to claim 1, wherein the hardware processor is further configured to obtain an input corresponding to a user-feedback associated with an effectiveness and an experience of the patient from the first rehabilitation therapy or the second rehabilitation therapy.

5. The system according to claim 4, wherein the hardware processor is further configured to update the second rehabilitation therapy to a third rehabilitation therapy based on the obtained input, wherein the input is one or more of: a gesture-based input, a voice-based input, or an input received via an input device that is communicatively coupled to the XR headset.

6. The system according to claim 1, wherein the pose information comprises one or more of: a position of the patient, an orientation of the patient in a three-dimensional (3D) space, a joint position of the patient in the 3D space, or other pose information.

7. The system according to claim 1, further comprising an artificial intelligence (AI) sub-system, wherein the hardware processor is further configured to learn, by use of the AI sub-system, a user-feedback and a performance gap specific to the patient from movement information corresponding to the tracked physical movements of the patient when the patient performs the first activity and the second activity indicated by the first rehabilitation therapy and the second rehabilitation therapy.

8. The system according to claim 7, wherein the hardware processor is further configured to dynamically update a current rehabilitation therapy to a new rehabilitation therapy, based on the performance gap learnt by the AI sub-system, wherein the current rehabilitation therapy is one of the first rehabilitation therapy or the second rehabilitation therapy.

9. The system according to claim 7, wherein the hardware processor is further configured to present a user interface to an electronic device associated with a care giver of the patient.

10. The system according to claim 9, wherein the hardware processor is further configured to obtain one or more new rehabilitation therapies based on one or more input received via the user interface, wherein the one or more inputs comprises a plurality of defined parameters to configure the AI sub-system to define the one or more new rehabilitation therapies.

11. The system according to claim 1, wherein the virtual environment is a metaverse.

12. A computer implemented method for providing rehabilitation in a virtual environment, the computer implemented method comprising:
presenting, by an extended reality (XR) headset, a first rehabilitation therapy to a patient in the virtual environment;
tracking, by a sensing device, physical movements of the patient when the patient performs a first activity indicated by the first rehabilitation therapy; and
receiving, by a hardware processor, sensing data from the sensing device;
determining, by the hardware processor, pose information associated with the first activity of the patient based on the received sensing data;
determining, by the hardware processor, a performance metric associated with the physical movements of the patient in the first activity based on the determined pose information;
comparing, by the hardware processor, the performance metric with a reference metric to determine whether the patient has successfully performed one or more defined physical movements for a completion of the first activity indicated by the first rehabilitation therapy; and
changing, by the hardware processor, the first rehabilitation therapy to a second rehabilitation therapy based on a difference between the performance metric and the reference metric upon determining that the patient has unsuccessfully performed the one or more defined physical movements for the first activity.

13. The method according to claim 12, further comprising quantifying, by the hardware processor, a progress of the patient in the rehabilitation in one or more of: in the first rehabilitation therapy, in the second rehabilitation therapy, or from the first rehabilitation therapy to the second rehabilitation therapy.

14. The method according to claim 13, further comprising updating, by the hardware processor, a current rehabilitation therapy to a new rehabilitation therapy, based on the quantified progress of the patient in the rehabilitation, wherein the current rehabilitation therapy is one of the first rehabilitation therapy or the second rehabilitation therapy.

15. The method according to claim 12, further comprising obtaining, by the hardware processor, an input corresponding to a user-feedback associated with an effectiveness and an experience of the patient from the first rehabilitation therapy or the second rehabilitation therapy.

16. The method according to claim 15, further comprising updating, by the hardware processor, the second rehabilitation therapy to a third rehabilitation therapy based on the obtained the input, wherein the user input is one or more of: a gesture-based input, a voice-based input, or an input received via an input device that is communicatively coupled to the XR headset.

17. The method according to claim 12, further comprising learning, by the hardware processor, a performance gap specific to the patient from movement information corresponding to the tracked physical movements of the patient by use of an artificial intelligence (AI) sub-system when the patient performs the first activity and the second activity indicated by the first rehabilitation therapy and the second rehabilitation therapy.

18. The method according to claim 17, further comprising updating, by the hardware processor, a current rehabilitation therapy to a new rehabilitation therapy, based on the performance gap learnt by the AI sub-system, wherein the current rehabilitation therapy is one of the first rehabilitation therapy or the second rehabilitation therapy.

19. The method according to claim 17, further comprising presenting, by the hardware processor, a user interface to an electronic device associated with a care giver of the patient.
